(19) United States
(12) Patent Application Publication
(10) Pub. No.: US 2019/0358688 A1
(43) Pub. Date: Nov. 28, 2019

(54) Structural Fill-Materials from Solid Waste and Method to Produce

(71) Applicant: Louis Structures, LLC, Racine, WI (US)

(72) Inventors: Benjamin Horvat, Racine, WI (US); Stephen R. Lyons, Racine, WI (US)

(73) Assignee: Louis Structures, LLC, Racine, WI (US)

(21) Appl. No.: 16/158,038

(22) Filed: Oct. 11, 2018

Related U.S. Application Data
(63) Continuation-in-part of application No. 15/986,516, filed on May 22, 2018.

(51) Int. Cl. B09B 3/00; A61L 2/23; C04B 28/02; C04B 18/30

(52) U.S. Cl. CPC B09B 3/0083; A61L 2/23; E04H 17/16; C04B 18/305; C04B 28/021

(57) ABSTRACT

A method for converting solid waste to a solid fill-material in the form of a multiplicity of compacted pieces, the method comprising mechanically reducing solid-waste piece size to form a solid-waste stream, heating the solid-waste stream, adding an antimicrobial agent and a stabilizer to the solid-waste stream, and compacting the solid-waste stream.

### RELATED APPLICATIONS

This application is a continuation-in-part of U.S. patent application Ser. No. 15/986,516 filed on May 22, 2018.

### FIELD OF THE INVENTION

This invention relates generally to structural fill-materials and also to solutions to problems related to dealing with municipal solid waste.

### BACKGROUND OF THE INVENTION

A major societal concern is the mounting problem of what to do about vast and increasing amounts of municipal solid waste (MSW). Waste generation continues to grow faster than the population. The ever-increasing MSW streams pose challenges in handling and disposing, and there are enormous costs and myriad problems imposed on government and private entities.

In a typical arrangement for dealing with municipal solid waste, a private waste-management company carries out oversight and operation of a landfill. The economics of the arrangement generally involve a waste removal and disposal payment made by the community to the company based on the amount of solid waste collected and removed. Communities and companies competing with one another typically engage in bidding and negotiating to arrive at an agreement, with desiring contracts involving a sufficient on-going waste volume to maintain company profitability. While this business model has proved reliable in the past, the current pace of solid waste production means that both communities and companies need innovative approaches to waste management.

A great deal of the current research and innovation related to MSW management is focused on extending the life and performance of existing landfills. However, these sorts of efforts are generally shortsighted in that they tend to merely show how to extend the lives of landfills for a few years, rather than solve the underlying need for sustained economic development in our communities. What is needed are more and better systems and methods for dealing with MSW. Processing municipal waste in order to create usable products is one approach. In fact, entire industries exist based on processing MSW, and on finding and implementing various uses for processed MSW having purpose-driven characteristics. There remains a pressing need for systems and methods that effectively reduce the total volume of solid waste and increase the reuse of byproducts of MSW processing.

There is a very apparent and growing concern about being able to find suitable new landfill sites. Increases in rates of waste disposal and reduced public acceptance of constructing and operating traditionally-designed MSW facilities create a deficit of MSW landfill space that must constantly be addressed by government and solid-waste companies. Landfills are viewed as liabilities, both in the economic sense and the community-development sense. There is a need for a paradigm shift from the present landfill-as-liability point of view to more of a landfill-as-asset point of view.

There is a need for improved barrier-wall structures, such as wall structures for border usage aimed at minimizing improper or unauthorized border crossings and facilitating the guarding of a border, has been a matter of great interest, concern and controversy. Aside from the public controversies and debates based on differing philosophical viewpoints with respect to national sovereignty and security, there are very practical issues feeding the debates.

Perhaps most notable among the practical concerns are the projected high costs and the related matters of who should pay for a barrier wall, or how to fund a barrier-wall structure along much of the southern border of the United States. Other practical matters of concern are about the adequacy of some barrier-wall structures to fulfill their intended purposes, regulatory concerns, and even concerns related to aesthetics. On the latter point, while the Great Wall of China, for example, has long been a tourist attraction, very few people are advancing any similar thought with respect to a barrier wall along much of the southern border of the United States. The concerns are much more practical in nature, with cost and funding topping the list.

In addition to the above-described needs regarding border walls, an even larger need exists with respect to barrier walls of the sort used along highways for sound control and other purposes. Great lengths of such barrier walls are built, and it is therefore desirable to find cost-effective ways of building such walls.

Various objects of the invention are apparent from the above background discussion and will be apparent from the descriptions and illustrations of various aspects of the present invention which now follow.

### BRIEF DESCRIPTION OF THE INVENTION

The present invention is a method for converting solid waste to a solid fill-material in the form of a multiplicity of compacted pieces. The method comprises mechanically reducing solid-waste piece size to form a solid-waste stream, heating the solid-waste stream, adding an antimicrobial agent and a stabilizer to the solid-waste stream, and compacting the solid-waste stream into the multiplicity of compacted pieces.

The term “solid waste” as used herein means solid waste such as municipal solid waste (MSW), commonly referred to as trash or garbage and consisting of everyday items discarded by the public, either alone or supplemented by waste materials of other sorts allowing processing in the ways described herein. The processing of such solid waste, to produce what is referred to herein by the term “processed solid waste,” is described later in this document. Such solid-waste processing to create useful stable solid fill-material is an alternative to placing solid waste into landfills; i.e., recently-collected MSW is what is converted. However, the solid-waste processing described herein can instead convert solid-waste removed from landfills in order to create the useful stable solid fill-material.

In preferred embodiments of the inventive method, the antimicrobial agent and the stabilizer are a single material selected from the group consisting of fly ash, lime, and a fly ash/lime mixture. In some of these embodiments, the weight ratio of the antimicrobial and stabilizer material to the solid waste is at least 5%, more particularly, the weight ratio of the antimicrobial and stabilizer material to the solid waste may be about 5-15%.

In highly-preferred embodiments, the antimicrobial and stabilizer material is the fly ash/lime mixture, and the fly ash/lime mixture is from about 15-70% fly ash by weight. In some of these embodiments, the fly ash/lime mixture is from about 50-60% fly ash by weight. Also, in some of these embodiments, the fly ash/lime mixture is preblended.

Some highly-preferred embodiments of the inventive method include adding water to the solid-waste stream before the compacting step.

Some preferred embodiments include adding water in-situ at a jobsite, and in some of these embodiments, the added water is a portion of a fly ash/lime slurry.

Some embodiments of the inventive method may include one or more of the following method steps: (a) the step of compacting the solid-waste stream is pelletizing; (b) jobsite in-situ bulk-compacting of the multiplicity of compacted pieces; (c) adding activated charcoal to the solid-waste stream; and (d) removing disallowed materials prior to reducing solid-waste piece size.

In some embodiments, the solid waste has been removed from a landfill.

In another aspect of the invention, the present invention is a method for converting solid waste to a solid fill-material in the form of a multiplicity of compacted pieces, and the method comprises mechanically reducing solid-waste piece size, heating the solid waste, adding an antimicrobial agent and a stabilizer to the solid waste, and compacting the solid waste.

In yet another aspect, the present invention is a solid fill-material in the form of a multiplicity of compacted pieces prepared by a method comprising mechanically reducing solid-waste piece size, heating the solid waste, adding an antimicrobial agent and a stabilizer to the solid waste, and compacting the solid waste into the multiplicity of compacted pieces.

This invention involves a purpose-driven approach to two entirely separate sorts of problems, and serves to significantly reduce costs associated with major kinds of barrier-wall structures. While not wanting to be bound by theoretical considerations, this invention is based in part on the realization that appropriate processing of municipal solid waste can provide valuable backfill material for use in unique barrier-wall structures.

Equipment usable for each of the steps of the aforementioned method is available to facilitate practice of such method, and acceptable choices would be apparent to those familiar with the present invention. The steps of mechanically reducing solid-waste piece size may be carried out with a variety of equipment, such as equipment for cutting, chopping, shredding or coarse-grinding.

Certain terms used in this document will be fully understood by reference to the following definitions:

The term “disallowed materials” as used herein means waste items such as liquid paint, solvents, insecticides, unacceptably-large assembled manufactured devices, large metallic objects and anything else that would not be susceptible to the remaining steps of the process.

The term “buffering” as used herein means the process of storing a stream of material and later removing the stream from storage for purposes of later blending with another stream.

The term “pellet” as used herein means a small compacted piece of material without regard to the specific cross-sectional shape or aspect ratio; as used herein, pellets can have without limitation circular, rectangular or other cross-sections and be of varying lengths—typically no more than a few inches in largest dimension.

The term “jobsite” as used herein means the final location at which the fill-material produced by the inventive method disclosed herein is installed to serve as a solid fill-material.

The term “bulk-compacting” as used herein means the process by which a multiplicity of compacted pieces are further compacted by a process which acts on numerous compacted pieces at the same time. Bulk-compacting may include processes such as being rolled over by equipment such as road roller or soil compactor.

### BRIEF DESCRIPTION OF THE DRAWING FIGURES

FIG. 1 is a perspective drawing of one section of an embodiment of the barrier-wall structure of this invention. The drawing includes a cross-sectional view of the barrier-wall structure.

FIG. 2 is a cross-sectional drawing of the barrier-wall structure embodiment of FIG. 1.

FIG. 3 is an illustration of some compacted pieces of processed solid waste, such pieces being pellets of different sizes.

FIG. 4 is a cross-sectional drawing of an alternative embodiment of the barrier-wall structure of this invention, such alternative including tensioning cables secured to and extending between the parallel retaining walls.

FIG. 5 is a perspective drawing of an embodiment of a barrier-wall structure of this invention in which the structure includes a single retaining wall. The drawing includes a cross-sectional view of the barrier-wall structure.

FIG. 6 is a schematic block diagram of an embodiment of the inventive method disclosed herein to convert solid waste to a stable solid fill-material in the form of a multiplicity of compacted pieces.

FIG. 7 is a schematic diagram of an embodiment of the inventive method shown in FIG. 6 which extends the method embodiment of block diagram of FIG. 6 to include the in-situ steps of adding water or a fly ash/lime slurry and further bulk-compacting the solid-waste fill material.

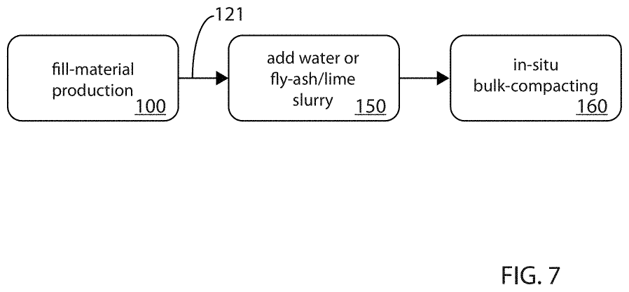

### DETAILED DESCRIPTIONS OF PREFERRED EMBODIMENTS

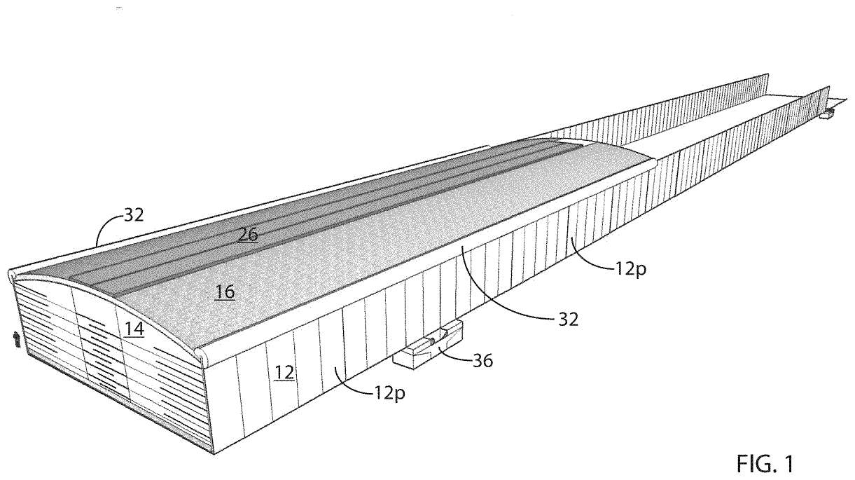

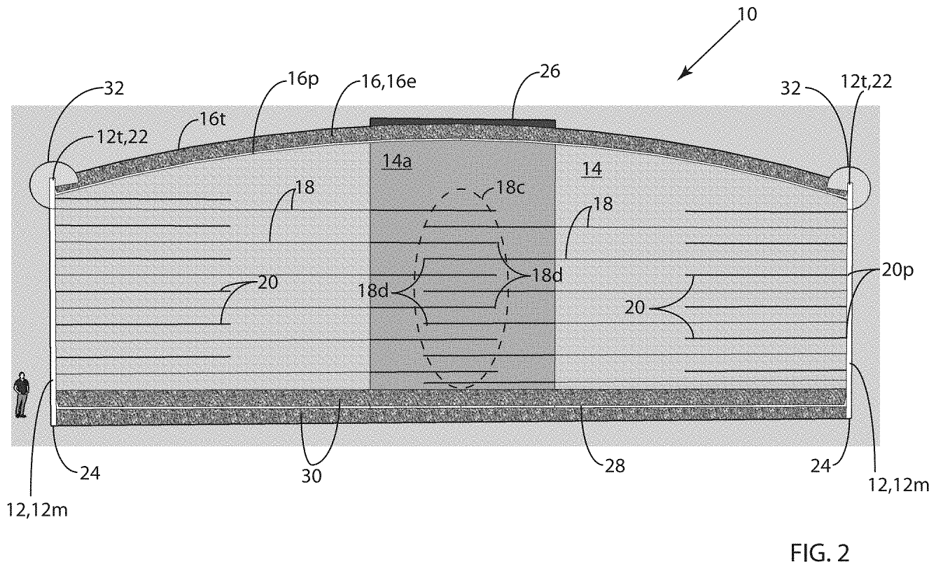

FIG. 1 is a perspective drawing of one section of an embodiment 10 of the barrier-wall structure of this invention, and FIG. 2 is a cross-sectional drawing of the barrier-wall structure embodiment of FIG. 1. Some reference numbers are more conveniently placed in one of FIGS. 1 and 2; please refer to these figures as necessary.

Barrier-wall structure embodiment 10 includes: (a) a pair of parallel retaining walls 12 which define a space between walls 12; (b) fill-material 14 within the space between walls 12, fill-material 14 including processed solid waste; and (c) a layered covering structure 16 over and supported by fill-material 14 and extending between parallel retaining walls 12. Each retaining wall 12 includes a multiplicity of pre-cast concrete wall panels 12p.

Embodiment 10 also includes two pluralities of vertically-spaced geosynthetic grid layers 18. Grid layers 18 of each plurality have a proximal edge 18p secured to one of the retaining walls 12, and grid layers 18 extend from wall 12 to which they are secured toward the other retaining wall 12. Grid layers 18 are vertically-spaced and have fill-material 14 between successive vertically-spaced geosynthetic grid layers 18. Suitable geosynthetic grid layers 18 are available at least from Tensar International Corporation of Alpharetta, Ga.

In embodiment 10 of the barrier-wall structure, fill-material 14 extends between the two retaining walls. Each retaining wall 12 has a ground-intersecting lower edge 24 (i.e., the line along retaining wall 12 at which the vertical plane of retaining wall 12 intersects the horizontal surface of the ground) and an upper-edge structure 22, and fill-material 14 extends between retaining walls 12, substantially filling the space from between ground-intersecting lower edges 24 and upper-edge structures 22. That is, in embodiment 10, the space between retaining walls 12 is entirely filled, or substantially entirely filled, with fill-material 14.

In embodiment 10, each of geosynthetic grid layers 18 has a distal edge 18d, and grid layers 18 of each plurality each extend toward the other retaining wall 12 to positions beyond distal edges 18d of grid layers 18 which extend from the other retaining wall 12 to form an overlapping region 18e which is particularly effective in providing stability to barrier-wall structure embodiment 10.

Embodiment 10 also includes a central portion 14a to which at least one stabilizing material (binder) has been added to enhance the mechanical properties of fill-material 14a, and distal edges 12d are located in central portion 14a. Fill-material 14a may contain additives such as cement or a polymeric binder; such list of additives is not intended to be limiting. Remaining filled space contains fill-material 14 which has not been enhanced with additive.

Embodiment 10 also includes two pluralities of vertically-spaced geosynthetic grid layers 20 which are shorter in length than grid layers 18. Grid layers 20 of each plurality have a proximal edge 20p secured to one of the retaining walls 12, and grid layers 20 extend from wall 12 to which they are secured toward the other retaining wall 12 but do not extend to form an overlapping configuration of grid layers like grid layers 18. Grid layers 20 are vertically-spaced and have fill-material 14 between successive vertically-spaced geosynthetic grid layers 20. It should be noted that other embodiments similar to embodiment 10 are possible such as embodiments which include either grid layers 18 or grid layers 20 but not both.

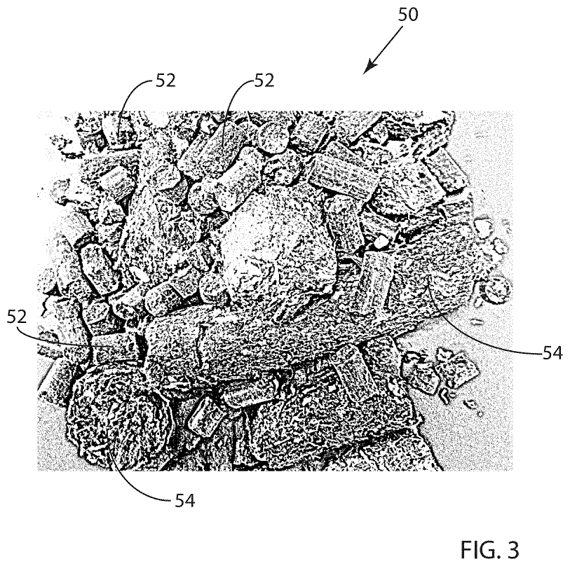

FIG. 3 is an illustration of a pile 50 of pellets 52, 54 which are compacted pieces of processed solid waste (fill-material 14). Pellets 52, 54 are of several different sizes such that pile 50 may be more effectively packed in the space between walls 12. This occurs because smaller pellets 52 can fit in the interstitial spaces between larger pellets 54. For example, larger pellets may be largest dimension of about five inches or less while the size(s) smaller pellets are chosen to assist in filling voids between pellets.

Fill-material 14 is processed such that a multiplicity of pieces together have a bulk density and friction angle acceptable for structural fill use. Preferably, the bulk density is within the range of about 115-130 pounds per cubic foot, and the friction angle is within the range of about 20-34 degrees, and most preferably within the range of about 28-34 degrees. Processed solid-waste fill-material 14 preferably meets the AASHTO LRFD criteria for a backfill promulgated by the United States Department of Transportation and similar specifications of other regulatory agencies. Fill-material 14 may also meet the criteria for a Beneficial Use Permit as promulgated by the United States Environmental Protection Agency.

Referring again to FIGS. 1 and 2, in embodiment 10, fill-material 14 and 14a is sufficiently compacted to permit vehicular transport on top of the wall structure. Layered covering structure 16 is upwardly convex with its uppermost portion parallel to retaining walls 12. Layered covering structure 16 includes a clay earthen layer 16e, a water-impervious polymeric-sheet layer 16p, and a drivable topmost layer 16t. Topmost layer 16t may be a spray-on concrete protective covering, and in embodiment 10 further includes an additional road portion over central portion 14a. Materials such as commonly-known 60 mil polyethylene sheet may be used for impervious polymeric-sheet layer 16p; such material is not intended to be limiting.

Barrier-wall structure embodiment 10 also includes a water-impervious liner 28 below fill-material 14,14a and sloping toward a collection point 36 (see FIG. 1). Bottom liner 28 serves to prevent any leachate from fill-material 14,14a from draining into the ground. The slope of bottom layer 28 may be about 2 degrees and directs leachate to collection point 36. (The slope of bottom layer 28 in embodiment 10 is down toward the right side of FIG. 1.) It should be noted that the inertness of fill-material 14,14a may eliminate the need for leachate handling. Bottom liner 28 is layered between two clay earthen layers 30.

In embodiment 10, each retaining wall 12 has a main wall portion 12m with a top edge 12t and an upper-edge structure 32. Upper-edge structure 32 is secured to top edge 12t and has outward and upward convexity which serves to frustrate efforts to use lines with grappling hooks to scale the wall and get on top of the barrier-wall structure. Such upper-edge structures 32 may include well-placed drainage holes (not shown in the cross-section of FIG. 2) to facilitate drainage of rain water and the like from barrier-wall structure embodiment 10.

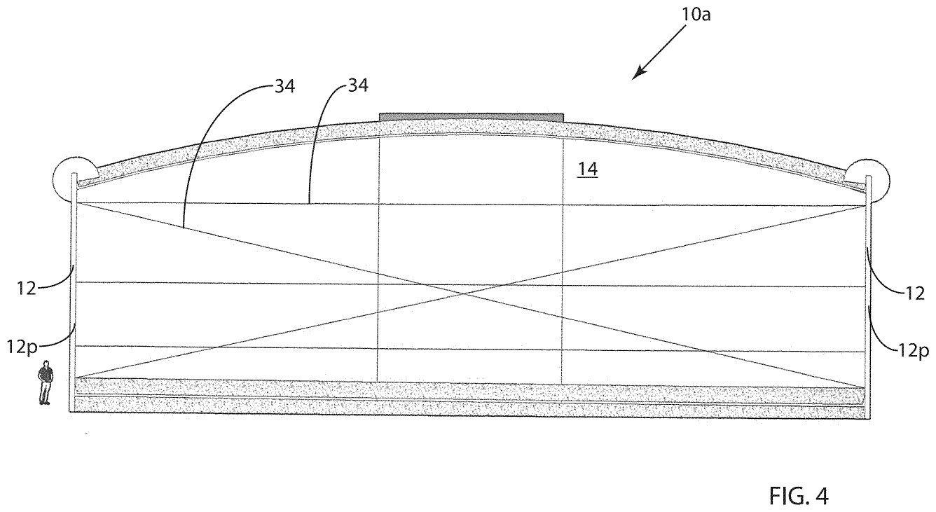

FIG. 4 is a cross-sectional drawing of an alternative embodiment 10a of the barrier-wall structure of this invention, such alternative including tensioning cables 34 secured to and extending between parallel retaining walls 12. FIG. 4 shows three generally horizontal cables and two diagonal cables; only two such cables 34 are labeled with reference numbers. Each tensioning cable 34 is attached at its opposite ends to pre-cast concrete panels 12p of parallel walls 12 and extends across the space between such parallel walls 12. In addition to providing enhanced stability to retaining walls 12, cables 34 may also effect some compaction of material-fill 14 after the filling of barrier-wall structure embodiment 10a with fill-material 14.

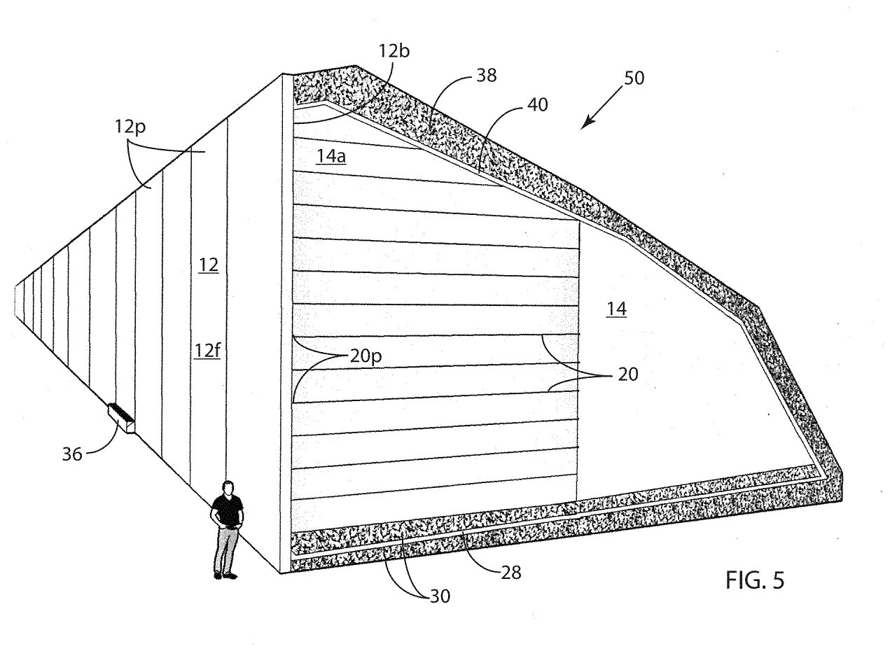

FIG. 5 is a cross-sectional drawing of an embodiment 50 of a barrier-wall structure of this invention in which the barrier-wall structure includes a single retaining wall 12 including pre-cast concrete panels 12p. Retaining wall 12 has a front surface 12f and a back support surface 12b. Back support surface 12b is supported by backfill which is processed solid-waste fill-material 14 and/or stabilizing-additive-containing fill-material 14a.

Single-wall embodiment 50 includes a plurality of vertically-spaced geosynthetic grid layers 20, and each of the grid layers has a proximal edge 20p secured at back support surface 12b and extending therefrom into fill-material 14a which is between vertically-spaced grid layers 20. Such configuration is not intended to be limiting; the backfill in embodiment 50 may be fill-material 14 or 14a or a combination thereof.

Embodiment 50 includes a top layer 38 and a water-impervious liner 40 on top of fill-material 14,14a. In similar fashion to embodiment 10, embodiment 50 also includes a water-impervious liner 28 below fill-material 14,14a and sloping toward collection point 36. Bottom liner 28 serves to prevent any leachate from fill-material 14,14a from draining into the ground. The slope of bottom layer 28 may be about 2 degrees and directs leachate to collection point 36. Bottom liner 28 is layered between two clay earthen layers 30.

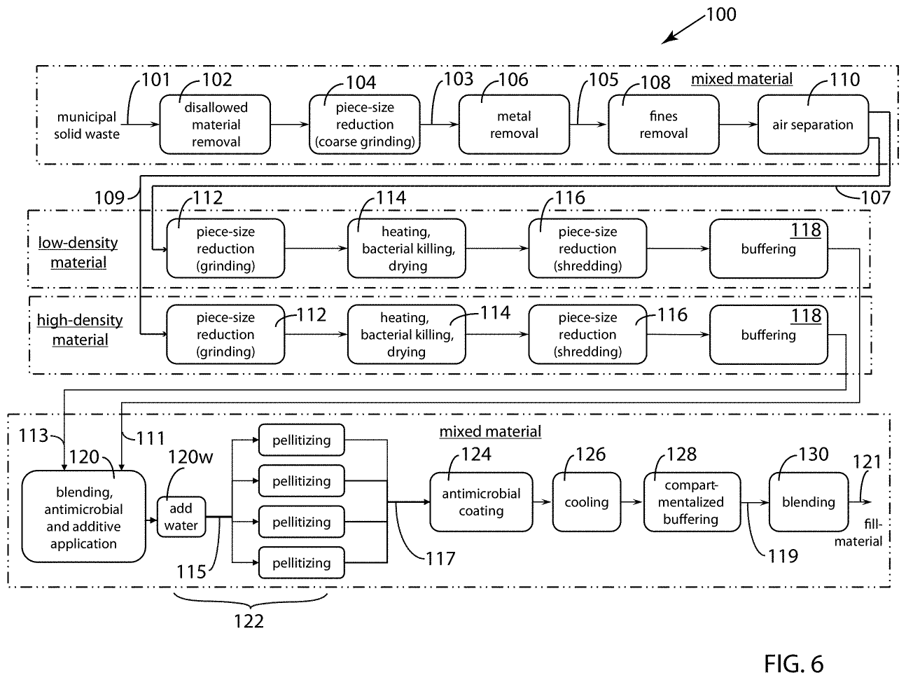

FIG. 6 is a schematic block diagram of an embodiment 100 of the inventive method disclosed herein to convert solid waste to a stable solid fill-material 14 in the form of a multiplicity of compacted pieces. The various steps of method embodiment 100 are often referred to below as “method elements.” As can be appreciated, some of the method elements included in embodiment 100 may be optional; modifications to embodiment 100 are possible.

An input stream 101 of MSW is entered into method embodiment 100 generally using trucks which haul the MSW immediately after collection or from a landfill. At method element 102, disallowed materials are removed from MSW 101. Disallowed materials may include but are not limited to objects such as propane tanks and containers of solvents. In method element 104, MSW 101 is then coarse-ground to reduce the piece size of the waste to be further processed, forming a first stream 103 of solid-waste. Piece sizes in first stream 103 may be about six inches or less in their largest dimension. This exemplary dimension is not intended to be limiting. At method step 106, both ferrous and non-ferrous metals are removed from first stream 103. Metal removal may be carried out using an eddy-current-sensor separator to form a second stream 105 of solid waste. Ferrous metal may also be removed using magnetic separation.

In method embodiment 100, fines which are present in second stream 105 are removed. In method step 110, second stream 105 is mechanically separated into a low-density stream 107 and a high density stream 109 using an air separation system. (The term “mechanically” is used here to describe the fact that the separation is done on the basis of density, a physical property of the solid waste rather than a chemical or electrical property of the solid waste. Therefore, as used herein, air separation is a mechanical process.)

In the next few method steps of embodiment 100, the separate streams 107 and 109 are processed separately, and method elements 112, 114, 116, and 118 are shown as being used to process streams 107 and 109 in this fashion. Although the specific equipment and/or process settings for the two streams may vary somewhat, the basic method elements are the same. Thus, in two method elements 112, low-density stream 107 and high-density stream 109 are separately ground to further reduce the piece size of the solid waste in streams 107 and 109. Such further-reduced piece sizes in streams 107 and 109 may be about 2 inches or less in their largest dimension. This exemplary dimension is not intended to be limiting. Streams 107 and 109 are then heated in method elements 114 to kill the bacteria present in streams 107 and 109, and drying may also occur depending on the moisture content of streams 107 and 109. Heating may be carried out by the application of steam. The killing of bacteria requires the solid waste material to reach a temperature at or above 160° F., but it is important that the temperature of streams 107 and 109 not be too much higher than that which is required for bacterial killing in order to prevent the melting of plastics which may be present therein.

The moisture content of streams 107 and 109 may vary considerably with variations in material content and weather. Thus, the amount of drying which may occur in method elements 114 may vary a large amount over such a wide range moisture content.

In embodiment 100, low-density stream 107 and high-density stream 109 are ground separately to further reduce the piece size of the solid waste in streams 107 and 109. Yet again further-reduced piece sizes in stream 107 and 109 may be about 0.5 inches or less in their largest dimension. As earlier indicated, exemplary dimensions are not intended to be limiting. As alluded to above, method elements 116 may be considered to be optional elements of the method depending on the needs of processing conditions. Then, method elements 118 represent the separate buffering of low-density stream 107 and high-density stream 109. Buffering 118 serves to smooth out the variations in the rate and content of solid-waste flow and permits control of the ratio of material from streams 107 and 109, to make adjustments to the density of a solid-waste stream 115 flowing out of the subsequent method element 120 by controlling the rates of flow in a low-density stream 111 and a high-density stream 113 flowing as inputs to method element 120.

In method element 120, the solid waste in streams 111 and 113 are blended and an antimicrobial agent is added to further kill bacteria and other living organisms and to control such organisms long-term. An antimicrobial as such from the Kathon™ product family of biocide chemicals available from Dow Chemical Company of Midland, Mich., may be used; such antimicrobial agent is not intended to be limiting; other such agents are also candidates for usage in fill-material 14. In method element 120, additives such as fill-material stabilizers to produce fill-material 14a and/or activated charcoal are also inserted into the processed solid waste. Fill-material stabilizers such as but not limited to cement and polymeric binders may be used. Activated charcoal may be used to sequester any leachate which may be present within fill-material 14.

The use of inexpensive antimicrobial agents and stabilizers is advantageous, and fly ash, lime, and a fly ash/lime mixture are particularly suitable additives. Due to the chemical makeup of both fly ash and lime, each of these materials, when wet, have both antimicrobial and stabilizing properties, and in particular, there are further advantages when both fly ash and lime are in the inventive solid-waste fill-material. Both materials are quite inexpensive, and at least in the case of fly ash, may be available at no cost or even an entity may be paid to take such material away from its source.

When wet, both fly ash and lime are alkaline materials and as such, provide antimicrobial action on the solid waste. A primary purpose of the heating step is the elimination or at least partial reduction of microbial activity in the solid waste, and the addition of a microbial agent provides further and longer-term reduction of such activity.

As mentioned above, both fly ash and lime each also have stabilizing properties, but when used together, provide additional structural stability to the solid-waste fill-material. The chemical reactions and physical chemistry of the such materials, alone or together, are well-known to those skilled in the area of chemistry and need not be described in further detail herein.

Low-grade fly ash, preferably fly ash with low abrasion characteristics, is easily available as is low-grade industrial lime.

The introduction of water into the solid-waste fill-material serves to activate the various chemical and physical changes necessary to provide the antimicrobial and stabilizing actions of the additives. This may occur in more then one stage in the overall method, first within the compacted pieces and subsequently, when either water or a fly ash/lime slurry is added in-situ to the fill-material in bulk form. Such second addition serves to create further structural stability of the fill-material by contributing binding between the compacted pieces.

There are other additives/binders which may be beneficial to introduce into the fill-material in addition to lime and/or fly ash. These may include a sodium-based activator such as sodium hydroxide or other sodium-based compounds in small quantities to increase the mechanical durability of the fill-material. Other non-sodium-based additives also can provide similar properties to the fill-material. All such additives may be used to accelerate the development of the compressive strength of the fill-material, and these additives may be added either prior to formation of the compacted pieces, at a jobsite as part of a slurry, or at both such times in the manufacture and installation of the fill-material.

Next, at method elements 122 in method embodiment 100, solid-waste stream 115 is pelletized into a stream 117 of the multiple compacted pieces for fill-material 14 or 14a. Four pelletizers are shown processing stream 115 in parallel. The size of the pellets produced in the pelletizing step 122 may be the same in each of the pelletizers or may differ among the pelletizers. Different size pellets produces a final mix of pellets which enables smaller pellets to fit within the interstitial spaces around larger pellets. Pellets of different shapes may also be produced.

During pelletizing step 122, steam may be introduced into the blended material in order to kill any remaining bacterial spores.

Next, in method element 124, the pellets in stream 117 of solid waste are coated with additional antimicrobial agent. Stream 117 is then cooled in method element 126 from the heating which occurs within pelletizing step 122. In method element 128, stream 117 undergoes compartmentalized buffering to permit adjustment of the density and friction angle of an output stream 119 of pellets. Variations in the content of MSW input to embodiment 100 may occur daily or even more frequently, and buffering stream 117 into a number of parallel compartments for later blending provides another level of control to ensure that required material properties of fill-material 14, 14a are met.

Other embodiments of such final buffering may also be used such as a single large mixer which continually mixes the buffered material to smooth out the statistical variations in pellet material properties. The use of compartmentalized buffering is not intended to be limiting.

Finally, in method embodiment 100, pellets in stream 119 are blended in method step 130 and are ready for delivery as fill-material stream 121.

While the principles of this invention have been described in connection with specific embodiments, it should be understood clearly that these descriptions are made only by way of example and are not intended to limit the scope of the invention.

1. A method for converting solid waste to a solid fill-material in the form of a multiplicity of compacted pieces, the method comprising:
mechanically reducing solid-waste piece size to form a solid-waste stream;
heating the solid-waste stream;
adding an antimicrobial agent and stabilizer to the solid-waste stream; and
compacting the solid-waste stream into the multiplicity of compacted pieces.

2. The method of claim 1 wherein the antimicrobial agent and the stabilizer are a single material selected from the group consisting of fly ash, lime, and a fly ash/lime mixture.

3. The method of claim 2 wherein the weight ratio of the antimicrobial and stabilizer material to the solid waste is at least 5%.

4. The method of claim 3 wherein the weight ratio of the antimicrobial and stabilizer material to the solid waste is about 5-15%.

5. The method of claim 2 wherein the antimicrobial and stabilizer material is the fly ash/lime mixture, and the fly ash/lime mixture is from about 15-70% fly ash by weight.

6. The method of claim 5 wherein the fly ash/lime mixture is from about 50-60% fly ash by weight.

7. The method of claim 5 wherein the fly ash/lime mixture is preblended.

8. The method of claim 1 further including adding water to the solid-waste stream before the compacting step.

9. The method of claim 1 further including adding water in-situ at a jobsite.

10. The method of claim 9 wherein the added water is a portion of a fly ash/lime slurry.

11. The method of claim 1 wherein the compacting is pelletizing.

12. The method of claim 1 further including jobsite in-situ bulk-compacting of the multiplicity of compacted pieces.

13. The method of claim 1 further including adding activated charcoal to the solid-waste stream.

14. The method of claim 1 further including the step of removing disallowed materials prior to reducing solid-waste piece size.

15. The method of claim 1 wherein the solid waste has been removed from a landfill.

16. A method for converting solid waste to a solid fill-material in the form of a multiplicity of compacted pieces, the method comprising:
mechanically reducing solid-waste piece size;
heating the solid-waste;
adding an antimicrobial agent and stabilizer to the solid-waste; and
compacting the solid-waste.

17. The method of claim 16 wherein the antimicrobial agent and the stabilizer are a single material selected from the group consisting of fly ash, lime, and a fly ash/lime mixture.

18. The method of claim 17 wherein the weight ratio of the antimicrobial and stabilizer material to the solid waste is at least 5%.

19. The method of claim 18 wherein the weight ratio of the antimicrobial and stabilizer material to the solid waste is about 5-15%.

20. The method of claim 17 wherein the antimicrobial and stabilizer material is the fly ash/lime mixture, and the fly ash/lime mixture is from about 15-70% fly ash by weight.

21. The method of claim 20 wherein the fly ash/lime mixture is from about 50-60% fly ash by weight.

22. The method of claim 20 wherein the fly ash/lime mixture is preblended.

23. The method of claim 16 further including adding water to the solid waste before the compacting step.

24. The method of claim 16 further including adding water in-situ at a jobsite.

25. The method of claim 24 wherein the added water is a portion of a fly ash/lime slurry.

26. The method of claim 16 wherein the compacting is pelletizing.

27. The method of claim 16 further including jobsite in-situ bulk-compacting of the multiplicity of compacted pieces.

28. The method of claim 16 further including adding activated charcoal to the solid waste.

29. The method of claim 16 further including the step of removing disallowed materials prior to reducing solid-waste piece size.

30. The method of claim 16 wherein the solid waste has been removed from a landfill.

31. A solid fill-material in the form of a multiplicity of compacted pieces prepared by a method comprising:
mechanically reducing solid-waste piece size;
heating the solid waste;
adding an antimicrobial agent and a stabilizer to the solid waste; and
compacting the solid waste into the multiplicity of compacted pieces.
